SEQUENCE LISTING (3 sequences)

SEQ ID NO: 1 - DNA, 22 bp, Salmonella enterica
  misc_feature: Fluor-labeled Probe
aaagtcggtctcgccgtcggtg

SEQ ID NO: 2 - DNA, 23 bp, Salmonella enterica
  misc_feature: Forward Primer
ctcaccaggagattacaacatgg

SEQ ID NO: 3 - DNA, 23 bp, Salmonella enterica
  misc_feature: Reverse Primer
agctcagaccaaaagtgaccatc

(19) United States
(12) Patent Application Publication
(10) Pub. No.: US 2011/0136105 A1
(43) Pub. Date: Jun. 9, 2011

(54) METHODS OF QUANTIFYING NUCLEIC ACIDS

(75) Inventors: Thomas W. Jantsch, Mission Hills, KS (US); Patrick Williams, Eudora, KS (US); Jennifer Gerst, Kansas City, MO (US)

(73) Assignee: Evogen, Inc., Kansas City, MO (US)

(21) Appl. No.: 12/960,967

(22) Filed: Dec. 6, 2010

Related U.S. Application Data
(60) Provisional application No. 61/267,035, filed on Dec. 5, 2009.

(51) Int. Cl. C12Q 1/68

(52) U.S. Cl. 435/6

(57) ABSTRACT

The present invention provides a method of detecting or quantifying a target nucleic acid by using fluorescently labeled oligonucleotide probes that do not rely on secondary structure or enzymatic action. The present invention provides improved methods of detecting or quantifying target nucleic acid by detecting or quantitating data during the annealing phase of PCR amplification.

### CROSS REFERENCE TO RELATED APPLICATIONS

This application claims priority from U.S. Provisional Application Ser. No. 61/267,035 filed on Dec. 5, 2009, which is hereby incorporated by reference in its entirety.

### BACKGROUND OF THE INVENTION

1. Field of the Invention

The present invention relates to methods for quantitating target nucleic acids during real time polymerase chain reaction amplification.

2. Description of the Related Art

A multitude of techniques for detecting specific nucleic acid sequences and scoring known single nucleotide polymorphisms have been described. Several of these detection methods utilize hybridization of fluorescently labeled probes or primers and rely on the transfer of energy between donor and acceptor moieties. Fluorescently labeled probes are typically single-stranded oligonucleotides that exhibit lower amounts of fluorescence emission in isolation than when hybridized to target sequences. The structures of these probes convey high specificity, permitting the identification of targets that differ by as little as a single nucleotide.

The energy absorbed by a fluorophore may be transferred to a quencher and released as heat. Quenching of fluorescent signal may occur by Fluorescence Resonance Energy Transfer (FRET) or non-FRET mechanisms. FRET quenching requires a spectral overlap between the donor and acceptor, where the efficiency of quenching is related to the distance between the two moieties. Non-FRET quenching occurs through short-range ‘contacts’ between fluorophore and quencher, requiring no spectral overlap between moieties.

A commonly used example that uses FRET quenching to analyze polymerase chain reaction (PCR) amplified target DNA are TaqMan™ assays. Taq Man probes are used PCR amplification to monitor the amplification of selected nucleic acid sequences. TaqMan™ probes have a 3′ quencher moiety and 5′ fluorescent reporter moiety and emit little fluorescence when intact due to efficient intra-molecular quenching. In a TaqMan™ assay, a probe specifically hybridizes to a segment of a target amplicon usually close to but not overlapping the primer on the 5′ end. As polymerase travels along in the 5′→3′ direction it encounters the TaqMan™ probe and cleaves the probe between the quencher and fluorophore moieties, thereby releasing the fluorophore, generally a reporter dye, into solution and yielding an increase in fluorescence. Fluorescence can be monitored during the extension phase of each PCR amplification cycle after sufficient target sequence has been generated to meet minimal detection limits.

TaqMan™ probes have high specificity and are designed to only bind to perfectly complementary sequence. Consequently, to detect single nucleotide polymorphisms (SNPs) multiple TaqMan™ probes are required as each probe can only detect a single SNP. It would be desirable to be able to detect multiple SNPs with a single probe to reduce the overall costs of detection. Because TaqMan™ probes are digested during PCR amplification, the probes are not available for post-amplification melting curve analysis. Further, because the probes are not cleaved until the extension phase of amplification, analysis cannot occur during the annealing phase of amplification.

An alternative to TaqMan™ probes are molecular beacons and eclipse probes. Molecular beacons are single-stranded oligonucleotide probes that are non-fluorescent in isolation, but become fluorescent upon hybridization to target sequences. Non-hybridized molecular beacons form stem-loop structures, possessing a fluorophore covalently linked to one end of the molecule and a quencher linked to the other, such that the hairpin of the beacon places the fluorophore moiety in close proximity with the quencher. When molecular beacons hybridize to target sequences, fluorophore and quencher moieties become spatially separated, such that the fluorophore is no longer quenched and the molecular beacon fluoresces. Molecular beacons may be employed in end-point and ‘real-time’ PCR assays for sequence detection and SNP discrimination. The secondary structure of the molecular beacon conveys high specificity to the hybridization probe, allowing the identification of targets that differ by a single nucleotide. However, the molecular beacon's intra-molecular interaction potentially produces a source of competition for inter-molecular target hybridization, and because the molecular beacon is an internal probe, it must compete with the amplicon's opposite strand for binding to the target sequence. The combination of both forms of competition may reduce molecular beacon hybridization efficiency to some target molecules. It is desirable to reduce such potential background interaction.

In addition, each molecular beacon can only detect a single SNP; therefore, multiple molecular beacon probes must be used to detect variations of a single SNP, which can lead to an increase in costs. Because they are not degraded during PCR amplification, molecular beacon probes are capable of emitting a signal during melting curve analysis.

Eclipse probes include a short oligonucleotide that is complementary to a target sequence, a fluorophore moiety, a quencher moiety, and a minor groove binder (MGB). Eclipse probes also include three modified bases. They function similar to molecular beacons.

Alternatively, probes may be physically bound to a primer. The overall structure of the probe section is similar to a molecular beacon; however, the 3′ end of the probe section that contains the quencher is lengthened by adding a primer. The primer attaches to the target sequence and is amplified during PCR by a polymerase. But unlike the TaqMan™ probes, the hairpin loop within the probe section is broken during the melting phase, causing a triplex to form between the target sequence, amplicon, and probe sequences. At a certain point the bonds between amplicon and target are broken and new bonds are formed between probe and amplicon causing a release of fluorescence on the 5′ end of the probe. This fluorescence can be monitored in real time during the extension stage of PCR, however publications in which the signal was read at the end of the annealing stage have also been found. These probe/primer combinations, i.e. scorpions, are useful tools for relative quantification, end point analysis, and SNP detection. As with the previous chemistries SNP detection of more than 1 allele requires a separate scorpion primer for each variance thus increasing the overall cost for multiple SNP detections.

Light-Upon-eXtension (LUX) primers function in the same way as that of scorpion primer probes; however, their overall structure is much simpler. LUX primers have only a reporter fluorophore attached near the 3′end of a primer. A tail of 5-7 nucleotides is added to form a sharp hairpin secondary structure on the 5′end. The secondary structure acts as a quencher. During the annealing phase of PCR amplification, the LUX primer loses its secondary structure and attaches to its target DNA sequence. Polymerase binds and amplifies as it does with standard primers. After extension the double stranded DNA allows the reporter to emit a signal that can be detected in real time PCR. LUX primers are capable of providing real time quantification data and being used in melting curve analyses.

### SUMMARY OF THE INVENTION

The present invention provides a method of detecting or quantifying target nucleic acid by using fluorescently labeled oligonucleotide probes that do not rely on secondary structure or enzymatic action. Instead, these probes include a fluorophore attached to an internal base, preferably centrally located within the oligonucleotide. Further, the present invention overcomes limitations of known detection methods by detecting or quantifying target nucleic acid during the annealing phase of PCR amplification rather than the melting or extension phases.

The invention provides methods of detecting or quantifying a target nucleic acid in a sample comprising (a) combining (i) an oligonucleotide having no substantial secondary structure, at least one internally positioned nucleotide labeled with a fluorescein based reporter without a quencher, modified at its 3′ end to prevent chain extension during PCR amplification, and fully complementary to and able to hybridize to one allele of the target nucleic acid with (ii) a sample suspected of containing the target nucleic acid, and (iii) a solution comprising a PCR amplification buffer, PCR primers, and nucleotide bases suitable for PCR amplification; (b) conducting PCR amplification; and (c) measuring an increase in fluorescence emission during annealing, wherein the increase in fluorescence is indicative of the presence of the target nucleic acid.

Methods of the invention include the use of multiple oligonucleotides, each of which is capable of hybridizing to either a different target sequence, or a different SNP of a target sequence. These oligonucleotides may anneal at the same or different temperatures. Further, these oligonucleotides may be labeled with the same or different fluorescein based reporters. It is preferred that different reporters are used and that at least one is a fluorescein based reporter.

The invention also includes methods of using a single oligonucleotide that is labeled with a fluorescein based reporter and at least one other reporter. The invention is able to detect or quantitate a first target sequence by measuring an increase in emission by the fluorescein based reporter during annealing and detect or quantitate a second target sequence or SNP by measuring the second reporter either during annealing or optionally during the melting or extension phases, depending upon the nature of the second reporter.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the relative fluorescence emitted during successive amplification cycles using three dilutions of genomic DNA.

FIG. 2 is a standard curve of data presented in FIG. 1.

### DETAILED DESCRIPTION

This invention embodies a new and simple fluorescent hybridization probe detection system that does not rely on probe secondary structure or enzyme action. Interaction between these hybridization probes and their target sequences generates significant alterations in fluorescence emission. Variations in hybridization potential allow discrimination of polymorphic targets by the amount of fluorescence emission and the melting temperature of the probe/target duplexes. The probes are used to acquire quantitative data on nucleic acid sequences (DNA and RNA) during the annealing phase of PCR amplification.

The probes confer the ability to monitor, in real-time, the amplification and accumulation of nucleic acid sequences during the annealing phase of the polymerase chain reaction, which allows for the collection of real time quantitative data. The use of these probes to generate, acquire, and process real-time data during the annealing phase of PCR is a new and novel approach by which PCR-based quantitation of DNA and RNA sequences may be performed.

In real-time quantitative PCR experiments, amplification reactions are characterized by the point in time during cycling when amplification of a PCR product is first detected (the crossing threshold: Ct), rather than observing the amplification product at the end of the PCR process (end point). Quantitative PCR has been performed using the probes described in U.S. Pat. No. 7,348,141 and U.S. patent application Ser. No. 12/101,392, both of which are incorporated herein in their entirety.

The length of the probe (or HyBeacon) is such that it is suitable for hybridizing with a complementary polynucleotide target, to provide a stable hybrid whose melting temperature depends on the exact sequence of the target. Oligonucleotides containing less than 15 nucleotide residues in certain cases do not form sufficiently stable hybrids, particularly where the two hybridizing sequences are not precisely complementary. Oligonucleotides which are longer than about 30 nucleotide residues in certain cases form hybrids whose melting temperature is relatively insensitive to the possible presence of a single nucleotide mismatch. Nucleotide residues are usually derived from the naturally occurring nucleosides A, C, G and T. However nucleotide analogues may be used at one or more locations of a probe, such nucleotide analogues being modified, e.g. in the base portion and/or the sugar portion and/or the triphosphate link. Base modifications, such as propynyl dU (dT-analogue), and 2-amino dA (dA analogue), generally alter the hybridization properties and may make the use of oligonucleotides having less than 15 or more than 30 nucleotide residues attractive. Alternatively, oligonucleotides composed of or comprising peptide nucleic acid (PNA), locked nucleic acid (LNA), 2′-0-methyl RNA, phosphoramidate DNA, phosphorothioate DNA, methyl phosphonate DNA, phosphotriester DNA, or DNA base analogues may be employed to form more stable interactions with target sequences.

In a preferred embodiment, more than one reporter is included in the oligonucleotide of a probe. But, it is understood that only a single reporter may be included. Herein, a “reporter” is understood to include all suitable fluorophores as well as other types of labels such as chromophores, radiolabels, dyes, pigments, and the like that are known in the art. The only limitation to a reporter is that it be suitable for attachment within an oligonucleotide in a probe and be detectable using the methods of the invention.

In a hybridization beacon or probe according to the invention, the oligonucleotide preferably has a sequence fully complementary to one allele of a known polynucleotide target having a known polymorphism, e.g. a point mutation or a single base insertion or deletion (SNP). The site of polymorphism is preferably, though not essentially, located centrally within the oligonucleotide probe.

Alternatively, the hybridization beacon may be complementary to a known non-polymorphic polynucleotide target and may simply be used to detect that target. Also, the hybridization beacons may be used to study potentially polymorphic targets with unknown and uncharacterized polymorphisms. The possibility is envisaged of mapping the position and/or nature of unknown polymorphisms by differential hybridization as measured during the annealing phase.

It may be convenient to provide two or more hybridization beacons, one fully complementary to each allele of the SNP under investigation. Where each hybridization beacon carries a different fluorophore, it may be convenient to mix the probes in solution for analysis of homozygous and heterozygous targets. In the same way, a mixture of hybridization beacons complementary to the various alleles of several different SNPs may be used together in solution for multiplex analysis, provided that each is labeled with a spectrally distinct fluorophore.

In another aspect, this invention provides a method of investigating a polynucleotide target, which optionally has a known or suspected polymorphism, by providing two or more oligonucleotide probes together. At least one oligonucleotide probe, and preferably more than one oligonucleotide probe, is a hybridization beacon as described in U.S. Pat. No. 7,348,141 and that emits a detectable fluorescence during the annealing phase of amplification.

The polynucleotide target may be DNA, RNA, or cDNA, and is used in single-stranded form, or a DNA duplex may be probed to form a triplex. The polynucleotide target has a known polymorphism, preferably a SNP. The target is incubated under hybridizing conditions with an oligonucleotide probe, preferably a hybridization beacon as herein described. It is necessary that the hybrid generate a stronger fluorescence signal than the single-stranded oligonucleotide probe during the annealing phase of PCR.

Typically, the hybridization beacon has a sequence complementary, typically fully complementary, to one allele of the target polynucleotide. Use of fully complementary beacons allows differentiation between matched and mismatched hybridization. Suitably, each of two or more different hybridization beacons has a sequence complementary, ideally fully complementary, to a different allele of the target polynucleotide.

Generally, nomenclatures used in connection with, and techniques of, molecular biology, genetics, and protein and nucleic acid chemistry and hybridization described herein are those well known and commonly used in the art. Unless otherwise indicated herein, the methods and techniques of the present invention are generally performed according to conventional methods well known in the art and described in various general and more specific references that are cited and discussed throughout the present specification. Enzymatic reactions and purification techniques are performed according to manufacturer's specifications, as commonly accomplished in the art, or as described herein. The nomenclatures used in connection with, and the laboratory procedures and techniques of, analytical and synthetic organic chemistry described herein is those well known and commonly used in the art. Standard techniques are used for chemical syntheses, chemical analyses, nucleic acid preparation and formulation.

Herein, “nucleic acid”, “nucleic acid sequence”, “polynucleotide”, “target sequence” and the like refer to a DNA, RNA, or cDNA having two or more nucleotide bases covalently bound to each other unless otherwise stated.

Unless otherwise defined, all technical and scientific terms used herein have the same meaning as is commonly understood by one of skill in the art to which this invention belongs at the time of filing. If specifically defined, then the definition provided herein takes precedent over any dictionary or extrinsic definition. Further, unless otherwise required by context, singular terms shall include pluralities, and plural terms shall include the singular. Herein, the use of “or” means “and/or” unless stated otherwise. All patents and publications referred to herein are incorporated by reference.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Quantitation of Target Nucleic Acids During Annealing

Hybridization probes were designed and manufactured using the protocols described in U.S. Pat. No. 7,348,141, incorporated by reference, except that the following sequences were used: HyBeacons Fluor-labeled Probe-5′ AAA GTC GG(Fluor T) CTC GCC G(Fluor T)C GGT G (Phos) 3′ (SEQ ID NO: 1); Forward Primer-5′ CTC ACC AGG AGA TTA CAA CAT GG 3′ (SEQ ID NO: 2); and Reverse Primer-5′ AGC TCA GAC CAA AAG TGA CCA TC 3′ (SEQ ID NO: 3). The fluorescent dyes used were selected from the group: 6-carboxyfluorsecein, tetrachlorofluorescein, and hexachlorofluorescein. But, it will be understood that any fluorescent dye suitable for attachment to a nucleotide base may be used.

Genomic DNA was extracted from Salmonella enterica subsp. enterica serovar typhi ATCC®167™ using Evogen ONE™ Lysis Buffer by following the manufacture's protocol for gram negative bacteria. The resulting nucleic acid was quantified using Invitrogen's Qubit fluorometer, and copy number was ascertained with the online conversion calculator by URI Genomic Sequencing Center (see Staroscik, Andrew, Online dsDNA calculator; available on the world wide web at uri.edu/research/gsc/resources/cndna.html, incorporated herein by reference). A PCR master mix was prepared on the 24× scale such that a 1× reaction of 20 μl contained 10 μl Promega Master Mix by Promega, 0.2 μl of 51 μM forward primer, 1.2 μl of 24 μM reverse primer, 2 μl of 3 μM HyBeacons™ probe, 2 μl of 1 of 3 dilutions of genomic DNA, and molecular grade water to 20 μl total. Both the primers and the HyBeacon™ probes were synthesized by atdbio, University of Southampton.

Thermal cycling and amplification monitoring was conducted on a Bio-Rad CFX 96 Real-Time thermal cycler. Cycling conditions consisted of an initial denature at 94° C. for 2 minutes followed by 40 cycles of 95° C. for 15 sec, 55° C. for 30 sec, and 72° C. for 30 sec. Real time data was acquired at the end of the annealing phase when the temperature was 55° C.

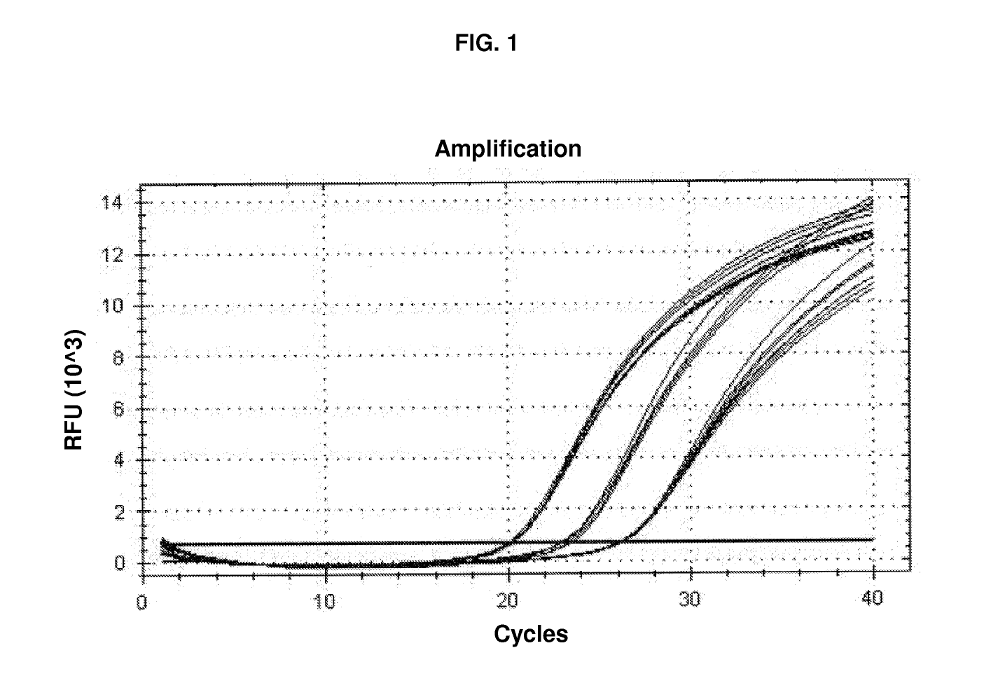

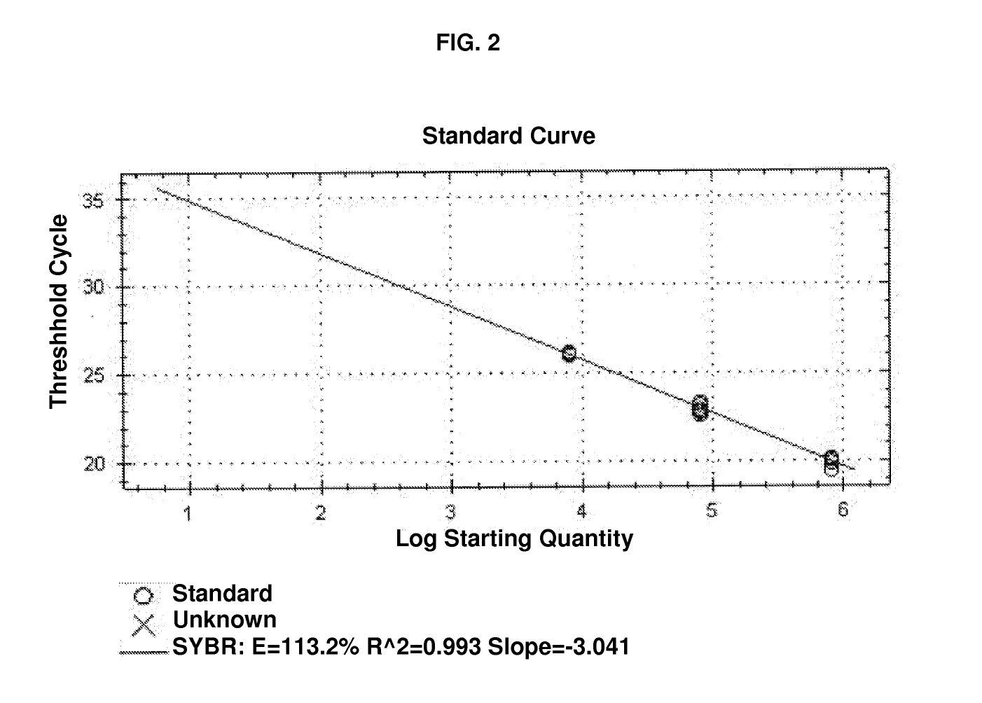

As shown in FIG. 1, samples with 8×10⁵ copies had an average crossing threshold (Ct) value of 20. Samples with 8×10⁴ and 8×10³ resulted in average Ct values of 22 and 24 respectively. A standard curve of data presented in FIG. 1 is shown in FIG. 2.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the following claims.

What is claimed is:

1. A method of detecting or quantifying a target nucleic acid in a sample comprising
a) combining an oligonucleotide having no substantial secondary structure, at least one internally positioned nucleotide labeled with a reporter, modified at its 3′ end to prevent chain extension during PCR amplification, and fully complementary to and hybridizes to one allele of the target nucleic acid with (i) a sample suspected of containing the target nucleic acid, and (ii) a solution comprising a PCR amplification buffer, primers, and nucleotide bases suitable for PCR amplification;
b) conducting PCR amplification; and
c) measuring the emission by the reporter during the annealing, wherein reporter demonstrates the presence of the target nucleic acid.

2. The method of claim 1, wherein the reporter is selected from the group consisting of fluorophores, chromophores, radiolabels, dyes, pigments, and combinations thereof, for labeling.

3. The method of claim 2, wherein the reporter is a fluorophores without a quencher, modified at its 3′ end to prevent chain extension during PCR amplification.

4. The method of claim 3, wherein the measuring is accomplished through demonstration of an increase in fluorescence emission during annealing, wherein the increase in fluorescence is indicative of the presence of the target nucleic acid.

5. The method of claim 1, wherein the detection or quantifying of the nucleic acid is conducted during the melting or extension phase.

6. The method of claim 1, wherein the method includes a first report and a second reporter on the labeled nucleotide.

7. The method of claim 6, wherein the emission by the first reporter is detected during the annealing phase and the emission by the second reporter is detected during the annealing phase, melting phase, or extension phase.

8. The method of claim 1, wherein the method includes more than one oligonucleotide.

9. A method of detecting or investigating a polynucleotide target comprising:
a) combining two oligonucleotide probes, a first oligonucleotide probe and a second oligonucleotide probe, with the first and second probe each having no substantial secondary structure, at least one internally positioned nucleotide labeled with a based reporter without a quencher, modified at its 3′ end to prevent chain extension during PCR amplification, and fully complementary to and hybridizes to a polynucleotide target with (i) a sample suspected of containing the polynucleotide target or polynucleotide targets, and (ii) a solution comprising a PCR amplification buffer, primers, and nucleotide bases suitable for PCR amplification;
b) conducting PCR amplification;
c) and measuring the emission of each reporter associated with the first and second oligonucleotide probes.

10. The method of claim 9, wherein the polynucleotide target is selected from the group consisting of DNA, RNA, cDNA, and combinations thereof.

11. The method of claim 9, wherein the polynucleotide target has a known polymorphism, wherein the polymorphism is a SNP.

12. The method of claim 9, wherein the reporter is selected from the group consisting of fluorophores, chromophores, radiolabels, dyes, pigments, and combinations thereof, for labeling

13. The method of claim 9, wherein the reporter of the first oligonucleotide probe is a fluorophores without a quencher, modified at its 3′ end to prevent chain extension during PCR amplification and wherein the measuring is accomplished through demonstration of an increase in fluorescence emission during annealing, wherein the increase in fluorescence is indicative of the presence of the target nucleic acid and the report of the second oligonucleotide is selected from the group consisting of fluorophores, chromophores, radiolabels, dyes, pigments, and combinations thereof.

14. A composition comprising:
a) a oligonucleotide having no substantial secondary structure, at least one internally positioned nucleotide labeled with a based reporter, modified at its 3′ end to prevent chain extension during PCR amplification, and fully complementary to and hybridizes to one allele of the target nucleic acid with (i) a sample suspected of containing the target nucleic acid, and (ii) a solution comprising a PCR amplification buffer, primers, and nucleotide bases suitable for PCR amplification.

15. A kit for use in detecting or quantifying a target nucleic acid, the kit comprising:
a) at least one oligonucleotide having no substantial secondary structure, at least one internally positioned nucleotide labeled with a based reporter, modified at its 3′ end to prevent chain extension during PCR amplification, and fully complementary to and hybridizes to one allele of the target nucleic acid; and
b) a solution comprising a PCR amplification buffer, primers, and nucleotide bases suitable for PCR amplification.

16. The kit of claim 15, wherein the kit includes an optional second oligonucleotide having no substantial secondary structure, at least one internally positioned nucleotide labeled with a based reporter, modified at its 3′ end to prevent chain extension during PCR amplification, and fully complementary to and hybridizes to one allele of the target nucleic acid.

17. The kit of claim 15, wherein the kit includes a vessel to combine at least one oligonucleotide with a sample suspected of containing the target nucleic acid and the solution to create a test solution.

18. The kit of claim 17, wherein PCR amplification is used on the test solution and the emissions from the reporter is measured during the annealing phase.

19. The kit of claim 18, wherein the emissions from the reporter is measured during the melting or extension phase.
